(19) United States
(12) Plant Patent Application Publication
(10) Pub. No.: US 2016/0270283 P1
(43) Pub. Date: Sep. 15, 2016

(54) Aeonium plant named 'Fiesta'

(50) ;

(71) Applicant: Renee O'Connell, Escondido, CA (US)

(72) Inventor: Renee O'Connell, Escondido, CA (US)

(21) Appl. No.: 14/544,911

(22) Filed: Mar. 6, 2015

(51) Int. Cl. A01H 5/00

(52) U.S. Cl. PLT/373

(57) ABSTRACT

A new and distinct Aeonium cultivar named ‘Fiesta’ is disclosed, characterized by a unique, stable, quadricolor variegation, displaying leaves of muted forest green, splashed with lime green, with rose red & burgundy blushing of the outer leaves of the rosette in high light environments, as well as during cooler winter temperatures. Additionally, the new cultivar ‘Fiesta’ is more easily and more rapidly propagated due to moderately fast, robust growth, enhancing production times. The new cultivar ‘Fiesta’ offsets freely at an early age, quickly forming attractive clumps, enhancing propagation intervals. The new variety is an Aeonium, part of the Crassulaceae complex that includes Aeonium, Echeveria, Pachyphytum, Graptopetalum, Sedum and others. Aeonium is a popular genus, typically produced as container plants for the patio or for use as landscape plants.

### LATIN NAME OF THE GENUS AND SPECIES

Aeonium hybrid

### VARIETY DENOMINATION

‘FIESTA’

### BACKGROUND OF THE INVENTION

The new cultivar, Aeonium ‘Fiesta’ , was found by the inventor, Renee O'Connell, as a naturally occurring, branch, mutation in an existing population of the unpatented unnamed, Aeonium cultivar referred to as Aeonium ‘#13’. The parent variety is the product product of a planned breeding program in a commercial greenhouse in Vista, Calif. conducted by inventor, Renee O'Connell. Aeonium ‘Fiesta’ was discovered by the inventor, Renee O Connell, in April of 2011, in a block of motherstock of Aeonium ‘#13’ at a commercial greenhouse in Vista. Calif.

Asexual reproduction of the new cultivar ‘Fiesta’ was first performed in Vista, Calif., at a commercial greenhouse, by vegetative cuttings in May of 2011. ‘Fiesta’ has since produced multiple generations and has shown that the unique features of this cultivar are stable and reproduced true to type.

### SUMMARY OF THE INVENTION

The cultivar ‘FIESTA’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘FIESTA’ These characteristics in combination distinguish ‘FIESTA’ as a new and distinct Aeonium cultivar:
1. Displays a unique, stable, quadricolor variegation, displaying leaves of muted forest green, splashed with lime green, with rose red & burgundy blushing of the outer leaves of the rosette in high light environments, as well as during cooler winter temperatures.
2. Offsets freely and forms attractive clusters at an earlier age than many other comparable Aeonium varieties.
3. Easily and rapidly propagated due to its robust growth; faster and easier to propagate than many other comparable Aeonium cultivars.
4. Freely offsetting, enhancing propagation and production.

Plants of the new cultivar ‘FIESTA’ are similar to plants of the parent, Aeonium ‘#13’ in most horticultural characteristics, however, plants of the new cultivar ‘FIESTA’ differs in the following;
1. Exhibits a unique, quadricolor variegation, displaying leaves of muted forest green splashed with lime green, with rose red and burgundy blushing of the outer leaves of the rosette in highlight environments, as well as during cooler winter temperatures, not exhibited by the Aeonium hybrid #13.
2. Exhibits a more compact morphology than the Aeonium hybrid ‘#13’.
3. More uniform arrangement of the leaves.

### COMMERCIAL COMPARISON

Plants of the new cultivar ‘FIESTA’ are comparable to the unpatented, commercial variety Aeonium arboreum ‘Tricolor’. The two Aeonium varieties are similar in most horticultural characteristics; however, the new variety ‘FIESTA’ differs in the following:
1. Displays a unique, quadricolor variegation, displaying leaves of muted forest green splashed with lime green, with rose red and burgundy blushing of the outer leaves of the rosette in high light environments, as well as during cooler winter temperatures. ‘Tricolor’ has an overall different variegation pattern, and does not develop the deep burgundy blushing to the outer foliage.
2. Freely produce offsets, producing an attractive, low-growing cluster, whereas Aeonium arboreum ‘Tricolor’ is a solitary plant until it attains a much taller height, and therefore cannot produce the same low growing, attractive cluster
3. By virtue of freely producing offsets, shortens the propagation and production interval, as compared with Aeonium arboreum ‘Tricolor’.

Plants of the new cultivar ‘FIESTA’ can also be comparable to the unpatented commercial variety Aeonium ‘Sunburst’ . The two Aeonium varieties are similar in most horticultural characteristics; however, the new variety ‘FIESTA’ differs in the following:
1. Different variegation pattern, and ‘Sunburst’ does not develop the burgundy coloration to outer leaves.
2. Exhibits early, profuse offsetting, not exhibited by Aeonium ‘Sunburst’.
3. Displays a more compact morphology than does Aeonium ‘Sunburst’.
4. By virtue of freely producing offsets, shortens propagation and production intervals, as compared to Aeonium ‘Sunburst’.

### BRIEF DESCRIPTION OF THE PHOTOGRAPHS

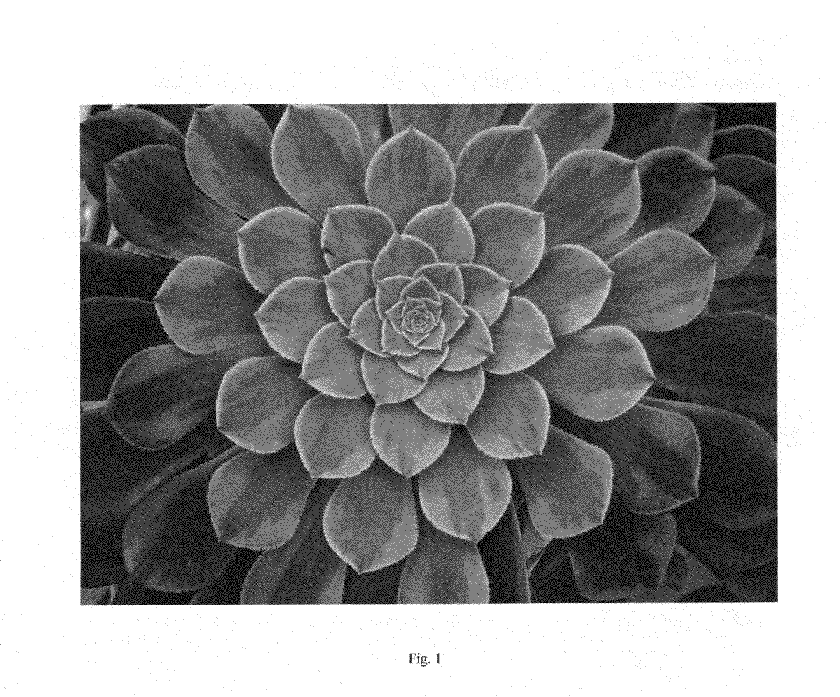

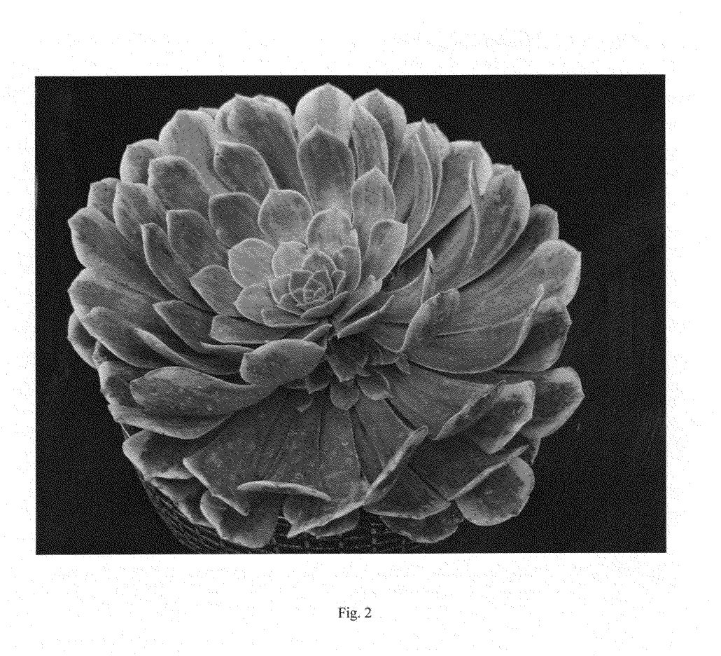

The accompanying photograph in FIG. 1 illustrates in full color the winter coloration of the new plant Aeonium ‘Fiesta’ as grown in a commercial greenhouse in Vista, Calif. FIG. 2 illustrates in full color the emergence of the offsets from between leaves of the rosette of Aeonium ‘Fiesta’ as grown in a commercial greenhouse in Vista. Plants illustrated in FIG. 2 have been grown in a shaded greenhouse, and the variegation is not well expressed. Age of the plants photographed is approximately 14 weeks from a rooted sideshoot plantlet. The photographs were taken using conventional techniques and although colors may appear different from actual colors due to light reflectance it is as accurate as possible by conventional photographic techniques. All photographs provided by the breeder.

### DETAILED BOTANICAL DESCRIPTION

In the following description, color references are made to the Pantone Process Color System Guide, Pantone CYMK, 2014, except where general terms of ordinary dictionary significance are used. The following observations and measurements describe ‘Fiesta’ plants in a commercial greenhouse in Vista, Calif. Temperatures ranged from −1° C. to 29° C. night and day. No artificial light, photoperiodic treatments or chemical treatments were given to the plants. Natural light conditions were approximately 2500 to 4000 fc of light. Measurements and numerical values represent averages of typical plant types.
Botanical classification.—Aeonium hybrid ‘FIESTA’.
Propagation:
   Type of propagation typically used.—Terminal vegetative divisions.
   Time to initiate roots.—About 19 days at approximately 23° C.
   Root description.—Fibrous.
Plant:
   Age of plant described.—Approximately 4 months.
   Container size of the plant described.—16 cm.
   Growth habit.—Upright, caulescent, with a terminal rosette, freely offsetting from below rosette and between leaves of the rosette to produce cluster.
   Height.—Approximately 10.7 cm to top of highest leaf. Height in cm to top of inflorescence is unknown as plant has been undergoing propagation, and has not yet flowered.
   Plant spread.—20.5 cm.
   Growth rate.—Moderately fast.
   Branching characteristics.—Freely offsetting.
Foliage:
   Leaf.—Arrangement: Rosulate. Average length: Approximately 8.5 cm. Longest length: Approximately 10.5 cm. Widest width: Approximately 3.1 cm. Width at base: 0.9 cm. Shape of blade: Oblanceolate. Apex: Acuminate. Base: Cuneate. Margin: Ciliate Texture of top surface: Glabrous Texture of bottom surface: Glabrous Quantity of leaves per plant: Approximately 95. Color: Young foliage upper side, margin: Near P 160-6 U Pantone. Young foliage, upper side, lighter green: Near P 159-5 U Pantone. Young foliage upper side, darker green: Near P 159-8 U Pantone. Young foliage, under side: Near P 159-8 U Pantone. Young foliage underside, towards stem: Near P 164-5 U Pantone. Mature foliage upper side, margin: Near P 160-6 U Pantone. Mature foliage upper side, lighter green: Near P 164-13 U Pantone. Mature foliage, upper side, darker green: Near P 159-15 U Pantone. Mature foliage under side, closer to apex: Near P 59-15 U Pantone. Mature foliage, under side, near stem: Near P 164-2 U Pantone. Mature foliage, under side, margin: Near P 160-13 U Pantone. Mature foliage, upper side, winter color, near apex: Near P 74-14 U Pantone. Mature foliage, upper side, winter color, other areas: Near P 74-4 U Pantone and P 74-16 U Pantone. Mature foliage, upper side, winter color, green area: Near P 159-13 U Pantone. Mature foliage, upper side, winter color, marginal cilia: Near P 7-9 U Pantone. Venation: There is no visual appearance of venation. Indistinguishable from leaf blade.
Flower:
   Natural flowering season.—Unknown, plant has not flowered to date.
Reproductive organs: Unknown, plant has not flowered to date.
Other characteristics:
   Fruits and seeds.—Unknown, plant has not flowered to date.
   Temperature tolerance.—Tolerates temperatures from approximately −2 C to 32 C.
   Disease/pest resistance.—Displays the same disease and pest resistance of any other comparable Aeonium.
   Drought tolerance.—Tolerates at least 3 weeks of high temperatures without supplemental water, showing no serious damage to plant.

What is claimed is:

1. A new and distinct cultivar of Aeonium plant named ‘FIESTA’ as herein illustrated and described.
